(19) United States
(12) Patent Application Publication
(10) Pub. No.: US 2024/0060027 A1
(43) Pub. Date: Feb. 22, 2024

(54) CELL CULTURE SCAFFOLD FORMED VIA 3D PRINTING

(71) Applicant: Copner Biotech Ltd, Abergavenny, Wales (GB)

(72) Inventors: Alan John COPNER, Abergavenny, Monmouthshire (GB); Jordan Callum COPNER, Abergavenny, Monmouthshire (GB)

(21) Appl. No.: 18/278,886

(22) PCT Filed: Feb. 18, 2022
(86) PCT No.: PCT/GB2022/000023

§ 371 Date: Aug. 25, 2023

(30) Foreign Application Priority Data
Mar. 14, 2021 (GB) 2103516.7
Oct. 1, 2021 (GB) 2114084.3

(51) Int. Cl. C12M 1/12; B29C 64/118

(52) U.S. Cl. CPC C12M 25/14; C12M 23/06; B29C 64/118

(57) ABSTRACT

Disclosed is a cell culture scaffold for in-vitro use, the scaffold comprising a series of porous cell growth walls, the series of walls being arranged in a generally concentric pattern each wall being spaced from its concentrically adjacent wall by an open channel suitable for nutrient supply. The scaffold is formed by a 3D printing printhead repeatedly forming a layer of single polymer strands in said pattern and repeatedly forming plural supports between each, or some of, the patterned layers, for spacing apart the or each patterned layer, whereby the walls have said porosity by virtue of the spacing of the or each patterned layer by the supports. Polyethylene terephthalate glycol is the preferred polymer.

### FIELD OF THE INVENTION

This invention relates to a bio-scaffold onto which cells can be seeded and multiplied and a method for manufacturing the bio-scaffold. The invention extends to software modelling of 3-dimensional small objects including, but not limited to, polymeric cell scaffolds to be employed in the field of mammalian cell culture research and manufacture by means of so-called 3D printing.

### BACKGROUND

Traditionally, mammalian cells are grown in a generally 2-dimensional environment, such as petri dishes, but this is not without its disadvantages, namely, the forced polarity that cells undergo due to the nature of the culture environment. Cells grown 3-dimensionally exhibit more physiologically relevant characteristics, for example, bone cell scaffolds can produce stronger bone grafting portions. Potentially, organ replacements or organ analogues could be cultured by means of a 3D scaffold.

The inventors have realised that successful tumour modelling can be achieved by seeding tumour cells onto the centre of a 3-dimensional scaffold and culturing those cells whilst monitoring their expansion and their adaptation as they multiply. Further, the inventor has found that what is needed is a scaffold which can mimic the regions of a tumour that have differing levels of oxygen/nutrient supply as the tumour grows, which in turn impacts the physiological conditions in which the tumour cells grow.

The inventors have further realised that cell culture scaffolds based on concentric constructs, such as circles, can provide consistent increasing nutrient supply channel size, emanating from the centre to the periphery of said scaffold. The effect of that increasing channel size is to provide distinct regions of the scaffold where cells will have more favourable nutrient and oxygen exchange (at the periphery) and areas not so (at the centre). Such a construct better represents physiological conditions in the body providing tangible experimental advantages for research in fields studying cell microenvironments, such as tumour micro-environment modelling in the field of cancer cell biology.

CN109266549A discloses an immune cell culture scaffold formed from stacked layers, each layer being formed from fibres woven together like the base of a willow basket. The fibres have holes laser-drilled to increase cell culture area. Whilst the disclosed construction appears to provide increased nutrient supply at its periphery by virtue of its construction, the woven nature of the construction would not provide reliable spacing and therefore is unlikely to be suitable for the repeatable experimental conditions required in biological research.

CN105056302A discloses a preparation method and application of a biological composite artificial trachea, by means of 3D printing a mixture including autologous cells onto a substrate, layer by layer to form a tube-like artificial trachea. The trachea is formed from rings of different materials which include cells, as well as radially extending spokes, however the purpose of the construct is to replace part of a trachea, not tumour modelling. There is no discussion of a gradient of intended nutrient supply within the artificial trachea.

Disclosed in “Engineered bone scaffolds with Dielectrophoresis-based patterning using 3D printing” by Zhijie Huan, Henry K. Chu, Hongbo Liu, lie Yang & Dong Sun and published in Biomedical Microdevices volume 19, Article number: 102 (2017), is stacked layers of cell scaffolds each consisting of generally concentric lamella rings which form a cathode and anode for dielectrophoresis purposes. The construct is individually layered and is weak due to the lack of internal interconnecting elements.

WO 20211086058 discloses an artificial tissue or organ analogue manufactured using three-dimensional cell printing. Concentric support rings are printed, and cells are introduced between the rings. That construct is stacked with similar constructs, leaving a space for nutrient supply between the layered constructs. There is no gradient of nutrient supply from the centre to the outside of the layered constructs.

### SUMMARY OF THE INVENTION

With the above in mind the inventors have devised an improved cell culture scaffold. The invention provides a cell culture scaffold for in-vitro use, the scaffold comprising a series of porous cell growth walls, the series of walls being arranged in a generally concentric pattern each wall being spaced from its concentrically adjacent wall by an open channel suitable for nutrient supply, said scaffold being formed by a 3D printing printhead repeatedly forming a layer of spaced single polymer strands in said pattern and repeatedly forming plural supports between each, or some of, the layers, for spacing apart the or each patterned layer, whereby the walls have said porosity by virtue of the spacing of the patterned layer(s) by the supports.

In an embodiment the supports extend generally radially and bridge respective concentrically spaced walls.

In an embodiment, portions of the polymer strands between adjacent supports are necked in cross section to enhance said porosity and increase the space between open channels.

In an embodiment, the concentric pattern is concentric rings, such as concentric circular rings, or regular nested polygons, such as hexagons or polygons with more sides, including dodecahedrons or irregular nested polygonal shapes with straight or curved sides or a combination of straight and curved sides.

In an embodiment, the bridging supports extend to, or through, the vertices of said polygons.

In an embodiment the cell growth walls when printed have a radial thickness in the range of 0.1 mm to 1 mm, and wherein optionally the channels are about the same width as the thickness of said walls.

In an embodiment, the diameter of the scaffold, or its greatest outer dimension is in the range of 5 to 50 mm, and/or its height is in the range of 1 to 25 mm.

In an embodiment the scaffold is formed from one, or a mixture of two or more of the following materials: polystyrene, polylactic acid, polycarbonate, polyethylene terephthalate glycol.

The invention further comprises a method for printing a cell growth scaffold for in-vitro use, the method comprising the steps of:
a) defining a wall layer of the scaffold including wall elements of the scaffold which, once combined with a multiplicity of similar wall layers would form a series of porous cell growth walls and defining a further layer which is intended to be printed between the, or some of, the multiplicity of wall layers; the series of walls being arranged in a generally concentric pattern each wall being spaced from its adjacent wall by an open channel suitable for nutrient supply,
b) preparing instructions for a 3D printer, including instructions in native Standard Tessellation Language (STL) data format for printing each layer and converting said in STL data into instructions suitable for operating a 3D printer; and
c) sending said instructions to a 3D printer and printing repeatedly a layer of spaced single polymer strands in said pattern and repeatedly forming plural supports between each, or some of, the layers, for spacing apart respective patterned layers, whereby the walls have said porosity by virtue of the spacing of the respective patterned layers, each wall being spaced from its adjacent wall by an open channel suitable for nutrient supply.

The invention extends to a method for in vitro culture of cells comprising preparing a cell culture scaffold according to the first aspect of the invention and seeding cells into the centre of the scaffold and culturing said cells, and optionally wherein the scaffold is seeded with no more than 50,000 cells, for example, following scaffold cleansing and hydration.

### BRIEF DESCRIPTION OF THE DRAWINGS

The invention will be better understood by way of the non-limiting examples described below, and with reference to the drawings, wherein:

FIG. 1 shows a plan view of one layout of a cell scaffold;

FIGS. 2 and 3 show a graphic representation of two adjacent layers of another cell scaffold;

FIG. 4 shows a graphic representation of layers shown in FIGS. 2 and 3 formed one on top of another to form multiple layers of a cell scaffold;

FIGS. 5, 6 and 7 show images of scaffolds as printed by means of a 3D polymer extrusion printer;

FIGS. 8 to 11 show graphic representations of alternative constructions of cell scaffolds; and

FIGS. 12 to 14 show magnified images of another alternative cell scaffold.

### DETAILED DESCRIPTION

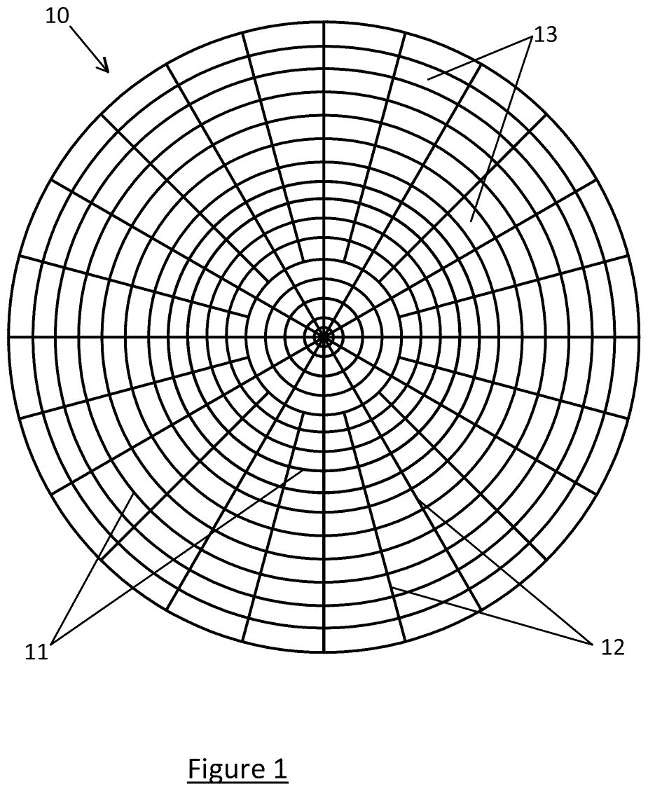

The concept of constructing scaffolds from layers is illustrated in FIG. 1; to be implemented using bespoke CAD modelling software. The modelling software graphically utilises rectangular primitives to design a 3D polymeric cell scaffold layer by layer; creating model constructs described directly in a file using Standard Tessellation Language (STL) data format. The layout of the cell scaffold 10 comprises plural concentric wall elements 11, in this case concentric circular elements, joined together by radially extending supports 12, the construct forming channels 13 which allow liquids to flow, for example for carrying nutrients and oxygen.

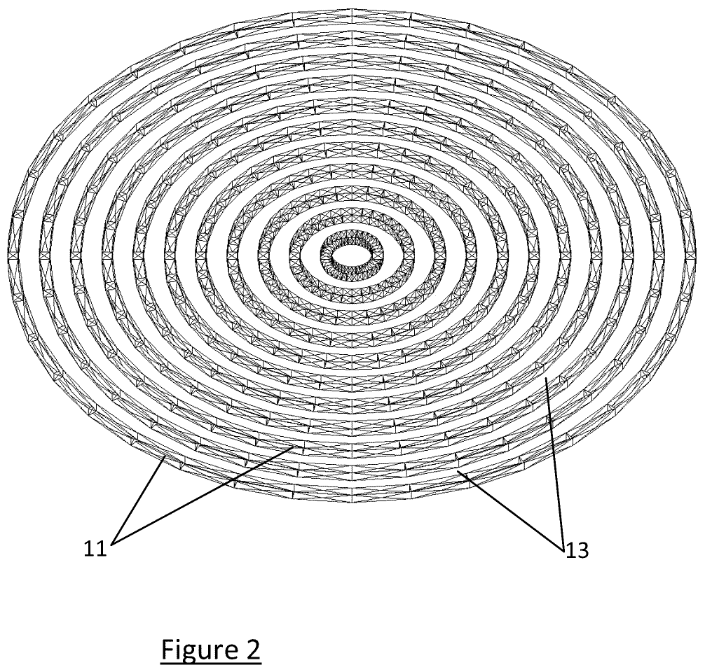

Referring to FIG. 2, one layer of a scaffold similar to that shown in FIG. 1 is graphically represented. A one-layer STL file will represent the wall elements 11, in this case having dimensions of: layer radial thickness 0.2 mm, height 0.2 mm, circle constructs radii 0.4 mm to 6.4 mm in increments of 0.6 mm, with open areas 13 therebetween which will form channels 13 once printed with other layers.

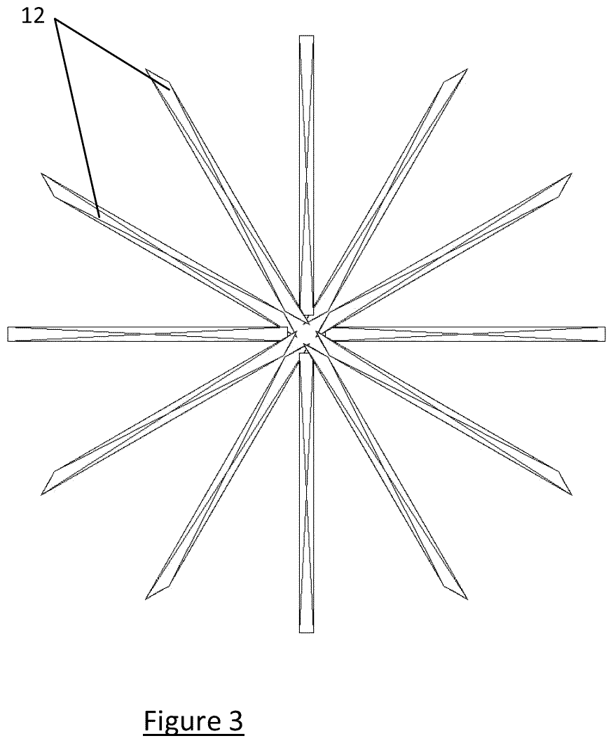

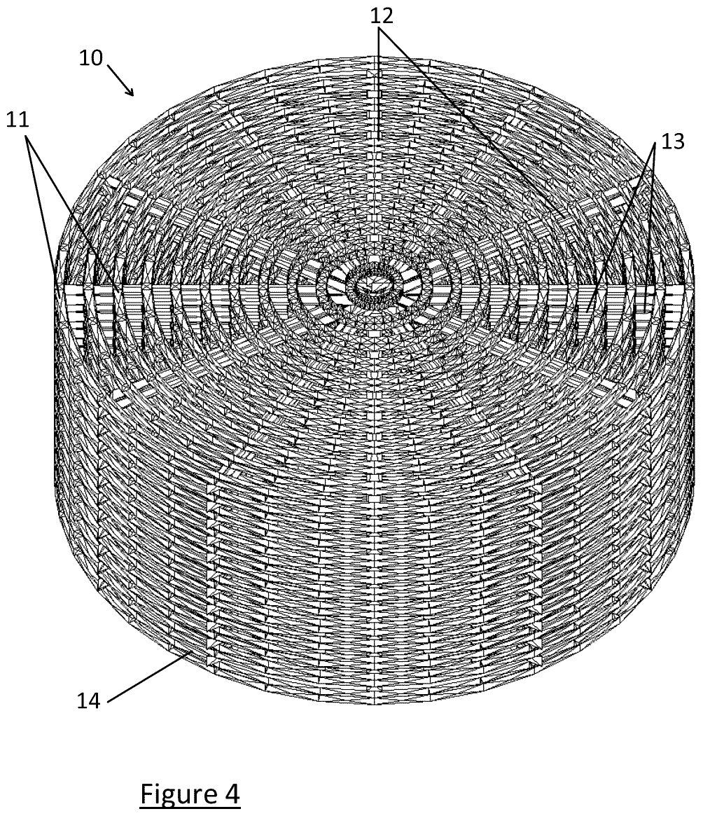

Referring to FIG. 3 another STL file will represent the radially extending supports 12 which space apart the layers depicted in FIG. 2 when printed. The dimensions in this case are: support constructs thickness 0.2 mm, height 0.3 mm, length 6.0 mm drawn at arc angles θ to 330 degrees at intervals of 30 degrees. These files can simply be repeatedly assembled using bespoke CAD modelling software (layer by layer)—walls (FIG. 2), spacer support (FIG. 3), walls, spacer support and so on to construct an overall cell scaffold model STL file according to the invention (e.g. FIG. 4). The overall cell scaffold model STL file (FIG. 4) is subsequently printed using a suitable plastics extrusion type printer. Where the wall elements 11 touch the supports 12, those plastics will fuse together to form a structure which is rigid and yet has open channels 13 and gaps 14 between adjacent elements 12 (FIG. 4).

The plastics extruded from the printing head is cool enough to inhibit substantial sagging of the plastics from one support 12 to another whilst printing takes place, such that the portion of the wall element plastics can bridge two adjacent supports without touching the element immediately underneath it as it is printed.

As more and more layers are printed a 3-dimensional cylindrical scaffold 10 can be formed, as graphically illustrated in FIG. 4, with an overall height of 4.8 mm and an external diameter of 13 mm, in this example. However, it has been found that with existing technology, scaffold heights of 1 mm and above, combined with diameters of 3 mm and above are readily achievable, and have most utility if they are sized to fit in the wells of standard multi-well laboratory plates.

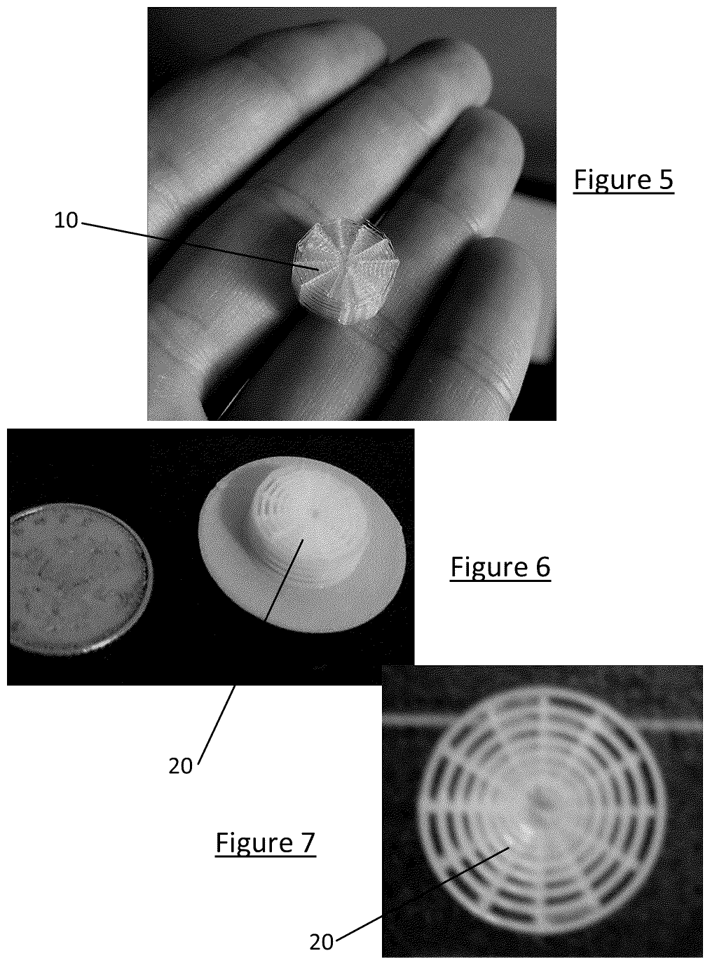

FIG. 5 shows a scaffold 10 according to the invention. The scaffold has been printed in the manner described above by means of a fused deposition modelling (i.e., extruding hot plastics from a print head moveable relative to the workpiece). The plastics material in this example is transparent polyethylene terephthalate glycol (PETG) which aids cell imaging in use because it is transparent. The printhead travel speed used was 10 mm per second, the rate of extrusion was 10 mm per second, the print nozzle temperature was 240 degrees Celsius, the bed temperature (the ambient temperature of the closed printing environment) was 70 degrees Celsius, the print nozzle diameter was 0.2 mm and the layer height was also 0.2 mm. These parameters allowed an extrusion of the plastics material which did not sag across a support layer where bridging was possible up to 4 mm without having any underlying support in the middle of the printed span.

FIGS. 6 and 7 show a scaffold 20 similar in construction to that of the scaffold 10 shown in FIG. 5, except that the scaffold 20 is formed from non-transparent polylactic acid (PLA) plastic material. PLA was used in this example because some cells which prefer growing on PLA, such as Chinese Hamster Ovarian (CHO) cells.

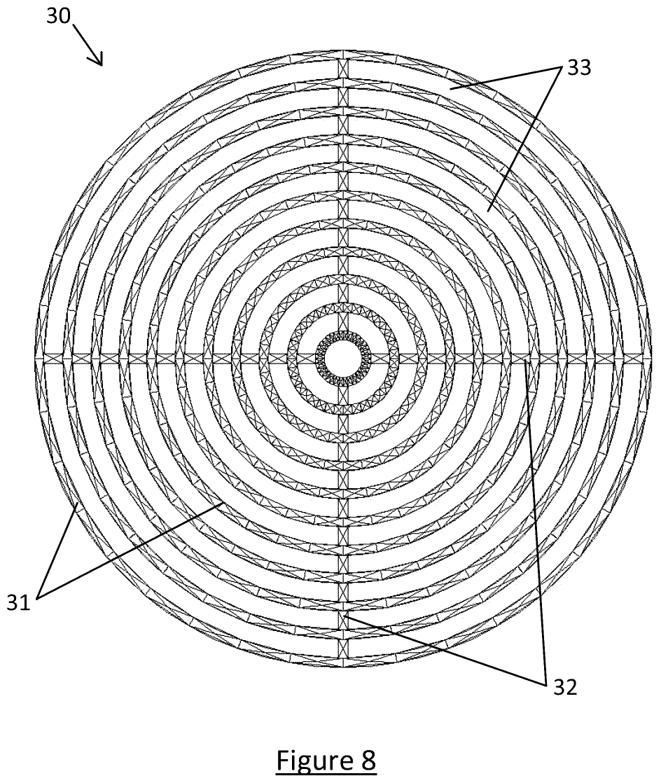

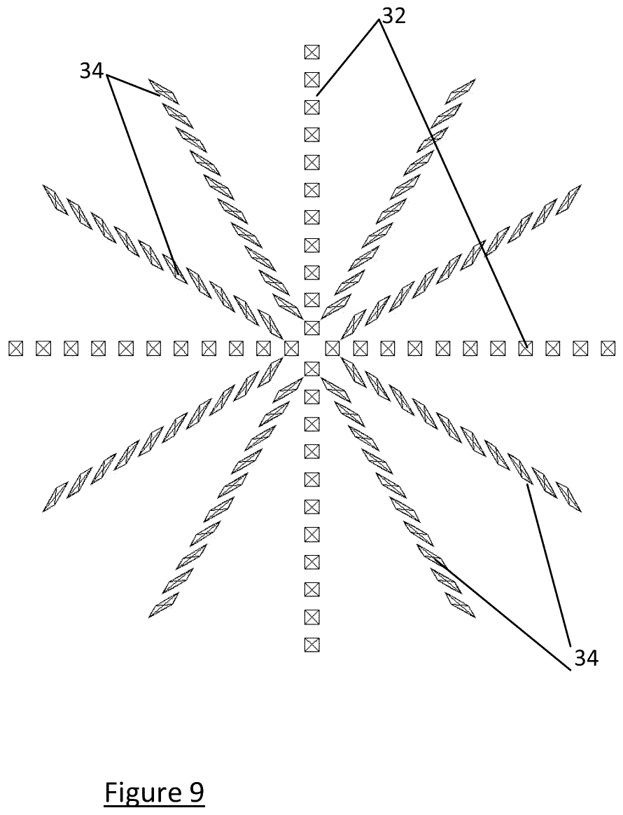

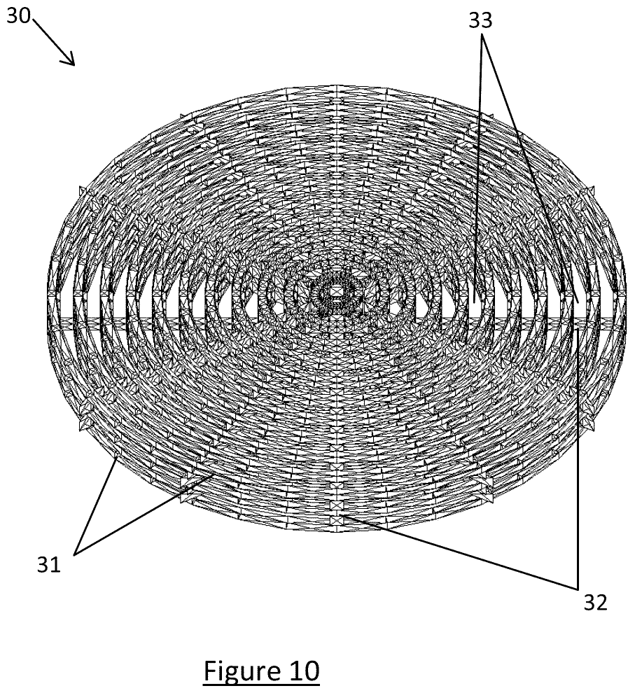

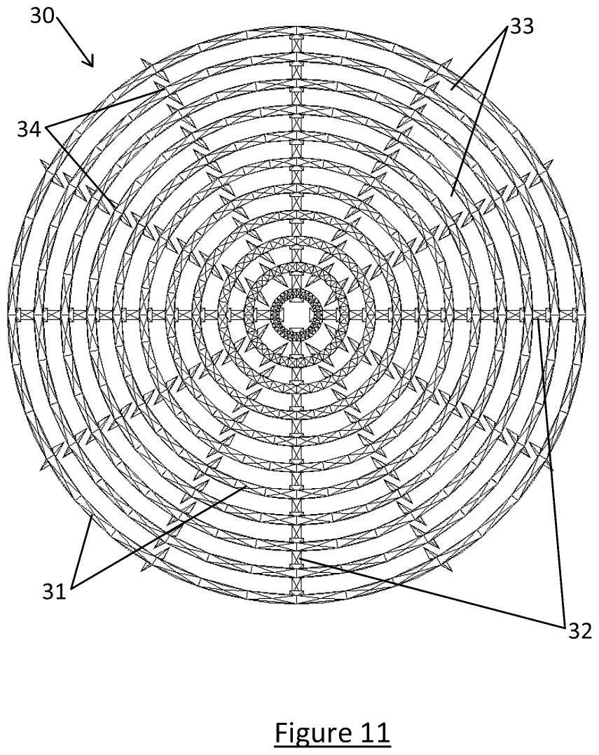

FIGS. 8 to 11 show a graphic representation of an alternative scaffold construction 30, where only 4 supports 32 extend radially outwardly to connect each of the concentrically arranged wall elements 31. In FIG. 1 it can be seen that there are twenty-four supports 12 (15 degrees apart), only half of which extend into the centre of the scaffold. In FIGS. 2 to 7 just twelve supports 12/22 are used (30 degrees apart). However, to maintain some rigidity and increase liquid flow in the channels 33, the elements 31 are further interconnected to each other and spaced by interconnecting struts 34 best shown in FIGS. 9 and 11, that do not extend substantially into the open channels 33, thereby increasing the area of the open channels 33. The dimensions of the scaffold are: wall elements 31 radial thickness 0.2 mm, height 0.2 mm, radii 0.4 mm to 6.4 mm in increments of 0.6 mm. Connecting spacer supports, thickness 0.2 mm, height 0.2 mm.

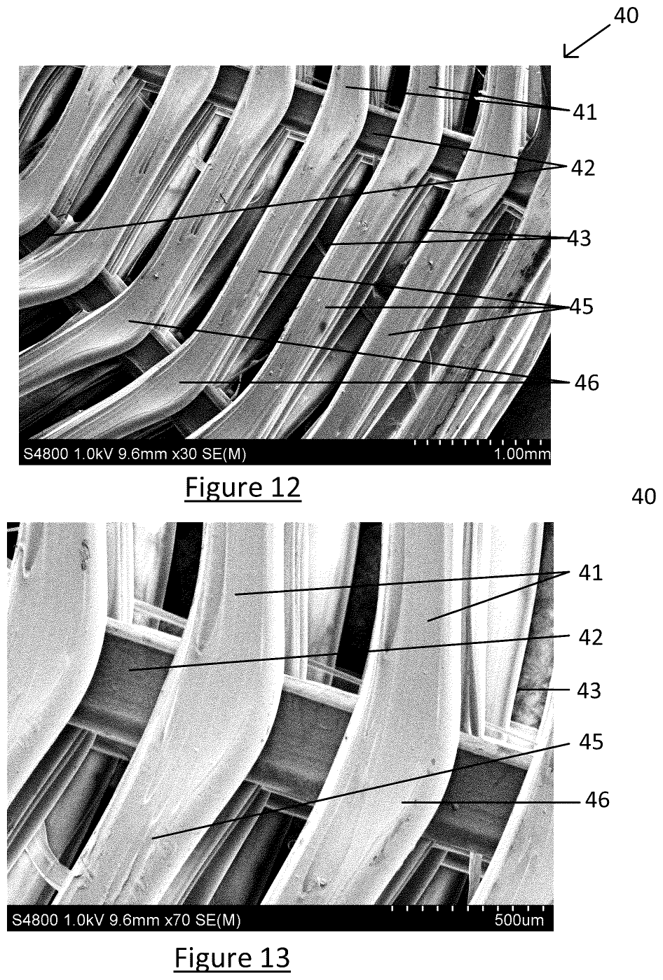

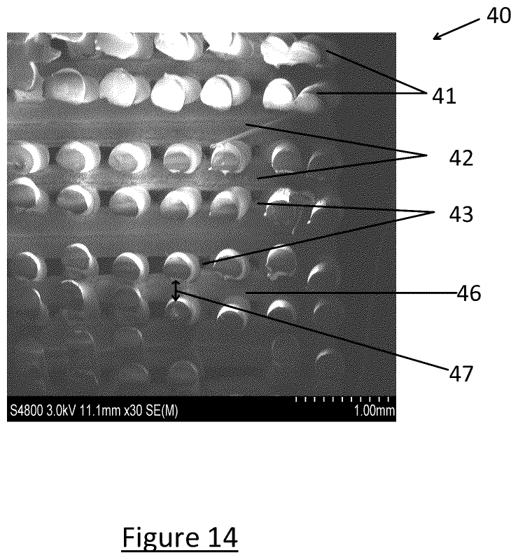

FIGS. 12 to 14 show enlarged images of a portions of a cell scaffold 40 printed as described above. The images show the wall elements 41 fused to supports 42 and spaced from each other by virtue of the intermediate supports 42. Another observation apparent from these enlarged images is the narrowing (necking) 45 and straightening of the wall elements between their respective support interconnections 46. This happens because the extruded plastics shrink once deposited and the shrinkage manifests itself as tension and necking between the relatively rigid supports. FIG. 14 shows a section through the wall elements 41, where it can be seen that the wall elements 41 do not touch, but rather are spaced by gaps 47 to provide a degree of porosity fed by open channels 43, which as mentioned above, are more free flowing as the channels increase in sectional area with increasing radius of the scaffold 40.

Also, more apparent in FIGS. 12 to 14 is the surface roughness and surface concavities in regions of the surface of the wall elements 41, most notably at the interconnections 46. These characteristics too are a result of the shrinking of the polymer after printing and provide regions where cells or cell clusters can pool when applied to a hydrated scaffold, and once colonised, the rough surface provides a more cell-adherent surface. Even if the scaffold is protein-coated, prior to cell seeding, the concavities will remain substantially, along with the surface roughness. Experimentation has shown that no more than 50,000 cells (typically 30,000-50,000) in suspension seeded to centre of the scaffold are sufficient to successfully start a tumour culture on the scaffold manufactured according to this invention, whereas commercial counterpart scaffolds require at least 250,000 cells for the same result. The reason for the relatively low number of seed cells is thought to be at least in part due to a combination of the open structure of the scaffold, and the pooling of cells in the concavities, as mentioned above, one under another, as the suspension of cells flows over the walls of the scaffold into successive underlying concavities when seeding takes place. The use of fewer cells reduces costs and reduces cell population stress due to apoptosis of redundant isolated cells, for example, those in the bottom of a receptacle used to house the scaffold.

In conclusion, the bio-scaffolds of the invention are formed from a three-dimensional construct consisting of repeated layer combinations constructed from concentric shapes layers, fixed together by spacing/support layers. Each concentric shape layer is formed of a collection of regular spaced concentric circle constructs (e.g. as depicted in FIG. 2), which serve to create open channels. Each support layer consists of a collection of radially spaced support constructs, traversing positional tangent points on the radii of the circle shape constructs defined in the circle shapes layer (e.g. as depicted in FIG. 3), beginning from the outer circle shape construct progressing to the inner circle shape construct. Each support construct within each support layer is drawn to be placed at 90 degrees angle to the concentric shapes layer adjacent to it.

Modelling software provides features that enable a designer to describe/draw scaffolds in the form of layers (employing individual STL layer files); with these layers being subsequently assembled to realise the scaffold model STL file. The software used utilises native Standard Tessellation Language (STL) data format both internally and outputting to a file, to accurately dictate how a 3D printer will render the scaffold constructs.

The printed scaffolds have a consistent structure, with heterogenous porosity. The total surface area and pore sizes exhibited by the scaffolds depend on the overall structure of the scaffold. This includes the number and size of the supports/struts within the support layers, the pattern in which the supports/struts are placed, the geometry of the concentric shapes layers, and the order and combinations of layers employed. Shape constructs within concentric shape layers can be spaced apart in a controlled fashion, as selected by the designer, in order to define the open channel dimensions between shape constructs.

The space/gap between concentric shape layers/patterns, and which provides said porosity as provided by the intervening support layers can range from small (50-100 microns) to large (100-400 microns) or a mixture of the two. The pore sizes within the complete scaffold vary, ranging anywhere from 10 to 1000 microns. The pores of the scaffold are all interconnected, providing an internal space for cells to infiltrate and adhere, either to one another, or the internal/external surface of the scaffold.

Examples of the bio-scaffolds proposed are manufactured in PETG plastic by a fused deposition modelling (FDM) 3D printer using the STL model file generated from the modelling process. Prior to printing the STL file is uploaded to slicer software, which converts the STL into a readable format for the 3D printer e.g., G code. For optimal printing performance printer settings are configured, such as hot melt chamber temperature, print bed temperature, extrusion speed, print head travelling speed and fan speed of the printer head, before finally uploading the G code file to the printer and printing the bio-scaffold as a single part. During printing each scaffold layer is deposited onto the former layer in a molten plastic form. On cooling, the scaffold layers will fuse, and thus, the bio-scaffold is formed in a layer-by-layer fashion.

Whilst various embodiments of the invention have been described and various dimensions, materials, and configurations have been suggested by way of example, it will be apparent to the skilled addressee that various additions, omissions, and modifications are possible without departing from the inventive concept defined by the claims herein. For example, the overall shape of the embodiments shown, ignoring the straightening of the wall elements due to shrinkage, is concentric circles, but other equally useful concentric patterns could be adopted, such as nested polyhedron patterns, or even irregular concentric patterns could be used, for example where the scaffold has to fit in an irregular receptacle. The preferred material for manufacture is polyethylene terephthalate glycol, or polylactic acid, but polystyrene, or polycarbonate, or combination thereof could be used to provide a suitable bio-scaffold. However, provided suitable printing parameters are used, any plastics polymer or polymers which occur in nature, or any mer which may polymerise during heating at the print head nozzle could be used. Porosity of the cell growth wall is provided, in the examples given above by gaps between layers, however the polymer material itself may provide a degree of liquid transmission which may increase the porosity of the walls still further.

1. A cell culture scaffold for in-vitro use, the scaffold comprising a series of porous cell growth walls, the series of walls being arranged in a generally concentric pattern each wall being spaced from its concentrically adjacent wall by an open channel suitable for nutrient supply, said scaffold being formed by a 3D printing printhead repeatedly forming a layer of single polymer strands in said pattern and repeatedly forming plural supports between each, or some of, the layers, for spacing apart the or each patterned layer, whereby the walls have said porosity by virtue of the spacing of the or each patterned layer by the supports.

2. The scaffold of claim 1 wherein the supports extend generally radially and bridge respective concentrically spaced walls.

3. The scaffold of claim 1, wherein portions of the polymer strands between adjacent supports are necked in cross section to enhance said porosity and increase the space between open channels.

4. The scaffold of claim 1, wherein the concentric pattern is concentric rings, such as concentric circular rings, or regular nested polygons, such as hexagons or polygons with more sides, including dodecahedrons or irregular nested polygonal shapes with straight or curved sides or a combination of straight and curved sides.

5. The scaffold of claim 1, wherein the bridging supports extend to, or through, the vertices of said polygons.

6. The scaffold of claim 1, wherein the cell growth walls when printed have a radial thickness in the range of 0.1 mm to 1 mm, and wherein optionally the channels are about the same width as the thickness of said walls.

7. The scaffold of claim 1, wherein the diameter of the scaffold, or its greatest outer dimension is in the range of 3 to 50 mm, and/or its height is in the range of 1 to 25 mm.

8. The scaffold of claim 1, wherein the scaffold is formed from one, or a mixture of two or more of the following: polystyrene, polylactic acid, polycarbonate, polyethylene terephthalate glycol.

9. A method for printing a cell growth scaffold for in-vitro use, the method comprising the steps of:
a) defining a wall layer of the scaffold including wall elements of the scaffold which, once combined with a multiplicity of similar wall layers would form a series of porous cell growth walls and defining a further layer which is intended to be printed between the, or some of, the multiplicity of wall layers; the series of walls being arranged in a generally concentric pattern each wall being spaced from its adjacent wall by an open channel suitable for nutrient supply,
b) preparing instructions for a 3D printer, including instructions in native Standard Tessellation Language data format for printing each layer and converting said in STL data into instructions suitable for operating a 3D printer; and
c) sending said instructions to a 3D printer and printing repeatedly a layer of spaced single polymer strands in said pattern and repeatedly forming plural supports between each, or some of, the layers, for spacing apart respective patterned layers, whereby the walls have said porosity by virtue of the spacing of the respective patterned layers, each wall being spaced from its adjacent wall by an open channel suitable for nutrient supply.

10. A method for in vitro culture of cells comprising preparing a cell culture scaffold, said scaffold comprising a series of porous cell growth walls, the series of walls being arranged in a generally concentric pattern each wall being spaced from its concentrically adjacent wall by an open channel suitable for nutrient supply, said scaffold being formed by a 3D printing printhead repeatedly forming a layer of single polymer strands in said pattern and repeatedly forming plural supports between each, or some of, the layers, for spacing apart the or each patterned layer, whereby the walls have said porosity by virtue of the spacing of the or each patterned layer by the supports, the method further comprising seeding cells into the centre of the scaffold and culturing said cells, optionally including cleansing and or hydrating said scaffold prior to said seeding.

11. A method according to claim 10 wherein the seeding step is seeding with a suspension of no more than 50,000 cells.

12. A method according to claim 9, wherein the STL data is graphically represented as rectangular primitives.

13. A method according to claim 12 wherein said rectangular primitives are as shown in any one or more of FIGS. 2,3,4,8,9,10 and/or 11.
